(19) United States
(12) Patent Application Publication
(10) Pub. No.: US 2017/0275225 A1
(43) Pub. Date: Sep. 28, 2017

(54) METHOD FOR THE PREPARATION OF 2-ALKOXY CYCLOHEXANOL

(71) Applicant: BASF SE, Ludwigshafen (DE)

(72) Inventors: Dominic Riedel, Lampertheim (DE); Joaquim Henrique TELES, Waldsee (DE); Andrei-Nicolae PARVULESCU, Ruppertsberg (DE); Stefan RÜDENAUER, Weinheim (DE); Thomas FENLON, Mannheim (DE)

(21) Appl. No.: 15/509,228

(22) PCT Filed: Sep. 8, 2015
(86) PCT No.: PCT/EP2015/070472

§ 371 Date: Mar. 7, 2017

(30) Foreign Application Priority Data
Sep. 9, 2014 (EP) 14184101.5

(51) Int. Cl. C07C 45/00; C07C 29/03; B01J 29/70; C07C 45/27; C01B 39/48; C07C 41/05; C07D 301/12

(52) U.S. Cl. CPC C07C 45/00; C07C 41/05; C07C 29/03; C07D 301/12; C07C 45/27; C01B 39/48; B01J 29/7038; C07C 2601/14; C07C 2601/16

(57) ABSTRACT

A method of preparing 2-alkoxycyclohexanol, a mixture comprising 2-alkoxycyclohexanol obtained via said method, and the use of said mixture for preparing 4-hydroxy-3-alkoxy-benzaldehyde.

The present invention relates to a method of preparing 2-alkoxycyclohexanol, to a mixture comprising 2-alkoxycyclohexanol obtained via said method, and to the use of said mixture for preparing 4-hydroxy-3-alkoxybenzaldehyde.

GB 2 252 556 A discloses a method of preparing 2-methoxy- and 2-ethoxycyclohexanols by reacting cyclohexene with hydrogen peroxide, methanol or, respectively, ethanol and optionally sulfuric acid in the presence of a catalyst composition prepared by drying and calcining a mixture of titanium tetraethoxide and silica gel in hexane or ethanol. Only when employed to prepare 2-methoxycyclohexanol does this method achieve a target product selectivity, for 2-methoxycyclohexanol, of 95%, albeit with the disadvantage that the admixture of sulfuric acid to the reaction mixture is required to achieve this high selectivity.

A. Corma et al., “Activity of Ti-Beta Catalyst for the Selective Oxidation of Alkenes and Alkanes”, Journal of Catalysis (1994), volume 145, pp. 151-158 discloses a method of preparing 2-alkoxycyclohexanol by reacting cyclohexene with hydrogen peroxide and an alkyl alcohol selected from the group consisting of methanol, ethanol and tert-butanol in the presence of a catalyst in the form of a Ti—Al-Beta zeolite. This method has the disadvantage that high excesses based on hydrogen peroxide are employed of cyclohexene and alkyl alcohol.

Y. Goa et al., “Catalytic Performance of [Ti, Al]-Beta in the Alkene Epoxidation Controlled by the Postsynthetic Ion Exchange”, Journal of Physical Chemistry B 2004, volume 108, pages 8401-8411 discloses a method of preparing 2-methoxycyclohexanol by reacting cyclohexene with hydrogen peroxide and methanol in the presence of a catalyst in the form of a Ti—Al-Beta zeolite. The ostensibly high selectivities reported for this method are based not on the target product, 2-methoxycyclohexanol, but on a mixture consisting of 1,2-cyclohexanediol and 2-methoxycyclohexanol.

E. G. Derouane et al., “Titanium-substituted zeolite beta: an efficient catalyst in the oxy-functionalisation of cyclic alkenes using hydrogen peroxide in organic solvents”, New. J. Chem., 1998, pages 797-799 discloses a method-of preparing 2-alkoxycyclohexanol by reacting cyclohexene with hydrogen peroxide and an alkyl alcohol selected from the group consisting of methanol, ethanol, propanol, isopropyl alcohol and tert-butanol in the presence of a catalyst in the form of a Ti—Al-Beta zeolite.

It is an object of the invention to provide a novel method of preparing 2-alkoxycyclohexanol.

It is another object of the invention to provide a mixture comprising 2-alkoxycyclohexanol.

We have found that, surprisingly, this object is achieved via a method for preparing 2-alkoxy-cyclohexanol in which a specific zeolitic material of framework structure MWW is employed as a catalyst having a high selectivity for the target product, 2-alkoxycyclohexanol.

The present invention accordingly provides a method of preparing a compound of formula (I)

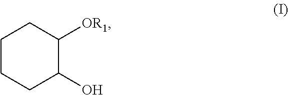

where R₁ is alkyl of 1 to 4 carbon atoms, comprising
(i) providing a liquid mixture comprising cyclohexene, an alcohol R₁OH, hydrogen peroxide and optionally a solvent;
(ii) reacting the cyclohexene with the hydrogen peroxide and the alcohol R₁OH, in the mixture provided as per (i) in the presence of a catalyst comprising a zeolitic material of framework structure MWW to obtain a mixture comprising the compound of formula) (I),

where the framework of the zeolitic material as per (ii) comprises silicon, titanium, boron, oxygen and hydrogen.

The present invention further provides a mixture comprising a compound of formula (I)

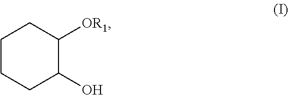

where R₁ is alkyl of 1 to 4 carbon atoms, obtainable or obtained as per that method of the present invention wherein the mole percentage for the compound of formula (I) in the mixture, based on the sum total of mole percentages for the compounds of formulae (I), (II), (III), (IV) and (V)

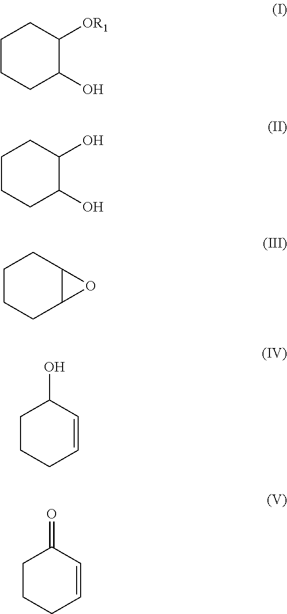

in the mixture is not less than 85%, preferably not less than 90%.

Step (i)

R₁ in the compound of formula (I) and the alcohol R₁OH is alkyl of 1 to 4 carbon atoms, i.e., of 1, 2, 3 or 4 carbon atoms. Step (i) permits the use of a mixture of two or more alcohols R₁OH that differ in alkyl R₁. R₁ may in principle be suitably substituted, in which case R₁ may have one or more substituents, which may each be, for example, hydroxyl, chloro, fluoro, bromo, iodo, nitro or amino. Alkyl R₁ is preferably unsubstituted alkyl, preferably selected from the group consisting of methyl, ethyl, n-propyl, isopropyl, n-butyl, isobutyl and tert-butyl, more preferably from the group consisting of methyl, ethyl, n-propyl, and isopropyl, more preferably from the group consisting of methyl and ethyl. R₁ is more preferably methyl.

The present invention accordingly also provides a method of preparing a compound of formula 0)

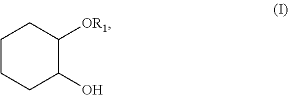

where R₁ is methyl, comprising
(i) providing a liquid mixture comprising cyclohexene, methanol, hydrogen peroxide and optionally a solvent;
(ii) reacting the cyclohexene with the hydrogen peroxide and the methanol in the mixture provided as per (i) in the presence of a catalyst comprising a zeolitic material of framework structure MWW to obtain a mixture comprising the compound of formula (I),

wherein the framework of the zeolitic material as per (ii) comprises silicon, titanium, boron, oxygen and hydrogen. The present invention further also provides a mixture comprising a compound of formula (I)

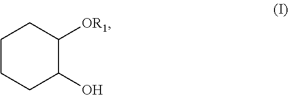

where R₁ is methyl, obtainable or obtained as per that method of the present invention wherein the mole percentage for the compound of formula (I) in the mixture, based on the sum total of mole percentages for the compounds of formulae (I), (II), (III), (IV) and (V)

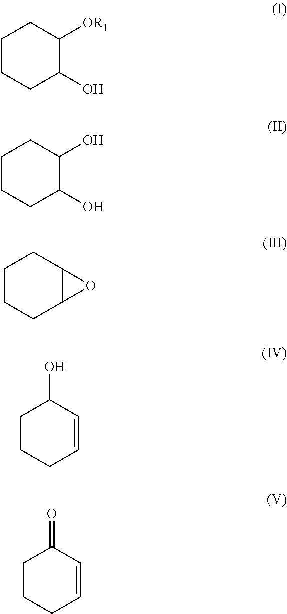

in the mixture is not less than 85%, preferably not less than 90%.

The composition of the liquid mixture as per (i) is in principle not subject to any special restriction.

In principle, the liquid mixture provided as per (i) may have a molar ratio prior to the reaction as per (ii) of cyclohexene:R₁OH less than, equal to or greater than 1:1. Preferably, the liquid mixture provided as per (i) has a molar ratio prior to the reaction as per (ii) of cyclohexene:R₁OH not more than 1:1. Further preferably the liquid mixture provided as per (i) has a molar ratio prior to the reaction as per (ii) of cyclohexene:R₁OH in the range from 1:1 to 1:50, more preferably from 1:3 to 1:30 and more preferably from 1:5 to 1:10.

In principle, the molar ratio of cyclohexene:hydrogen peroxide in the liquid mixture provided as per (i) may be less than, equal to or greater than 1:1 before the reaction of (ii). Preferably, the molar ratio of cyclohexene:hydrogen peroxide in the liquid mixture provided as per (i) is not less than 1:1 before the reaction of (ii). More preferably, the molar ratio of cyclohexene:hydrogen peroxide in the liquid mixture provided as per (i) is in the range from 1:1 to 5:1, more preferably from 1.5:1 to 4.5:1, more preferably from 2:1 to 4:1, before the reaction of (ii).

In principle, the liquid mixture provided as per (i) may comprise a solvent. Any solvent is preferably selected from the group consisting of C1-C6-alkyl nitriles, i.e., C1-, C2-, C3-, C4-, C5- or C6-alkyl nitriles, dialkyl ketones of the formula R₂—CO—R₃, where R₂ and R₃ are each independently selected from the group consisting of C1-C6-alkyl, i.e., C1-, C2-, C3-, C4-, C5- or C6-alkyl, and a mixture of two or more thereof, more preferably selected from the group consisting of C1-C3-alkyl nitriles, i.e., C1-, C2- or C3-alkyl nitriles, dialkyl ketones of the formula R₂—CO—R₃, where R₂ and R₃ are each independently selected from the group consisting of C1-C3-alkyl, i.e., C1-, C2- or C3-alkyl, and a mixture of two or more thereof, more preferably selected from the group consisting of acetonitrile, acetone and a mixture thereof.

When a solvent is included, the molar ratio of solvent:cyclohexene in the liquid mixture provided as per (i) may in principle be less than, equal to or greater than 1:1 prior to the reaction of (ii). Preferably, the molar ratio of solvent:cyclohexene in the liquid mixture provided as per (i) is not less than 1:1 before the reaction of (ii). More preferably, the molar ratio of solvent:cyclohexene in the liquid mixture provided as per (i) is in the range from 20:1 to 1:1, more preferably from 15:1 to 1:1, more preferably from 10:1 to 1:1, before the reaction of (ii). When the solvent in the mixture comprises a mixture of two or more solvents, the molar ratio of solvent:cyclohexene is based on the mixture of solvents.

The mixture provided as per (i) preferably comprises no solvent. In this case, the liquid mixture provided as per (i) is preferably not less than 90% by weight, more preferably not less than 95% by weight, more preferably not less than 98% by weight, more preferably not less than 99% by weight, more preferably not less than 99.5% by weight, more preferably not less than 99.9% by weight comprised of cyclohexene, R₁OH, methanol, hydrogen peroxide and any water from water being employed, as described below, in the form of an aqueous solution.

The temperature at which the liquid mixture is provided as per (i) is in principle not subject to any restriction. The liquid mixture as per (i) is preferably provided at a temperature in the range from 5 to 50° C., more preferably at a temperature in the range from 10 to 40° C., more preferably at a temperature in the range from 15 to 30° C.

The step of providing the liquid mixture as per (i) is in principle not subject to any special restriction. For instance, the liquid mixture as per (i) is providable by mixing the cyclohexene, the alcohol R₁OH, the hydrogen peroxide and optionally the solvent in any order. The liquid mixture as per (i) is preferably provided by admixing the hydrogen peroxide to a mixture comprising the cyclohexene, the alcohol R₁OH and optionally the solvent. It is preferable in this case for the mixture comprising the cyclohexene, the alcohol R₁OH and the optional solvent to be presented as the initial charge at a temperature in the range from 5 to 50° C., more preferably from 10 to 40° C., more preferably from 15 to 30° C., and for the temperature of the mixture resulting from admixing the hydrogen peroxide to be suitably maintained within the aforementioned temperature ranges.

The hydrogen peroxide is preferably admixed in the form of a solution in one or more suitable solvents. Possible solvents include, for example, water or organic solvents such as, for example, organic solvents selected from the group consisting of C1-C6-alcohols, C1-C6-alkyl nitriles, dialkyl ketones of the formula R₂—CO—R₃, where R₂ and R₃ are each independently selected from the group consisting of C1-C6-alkyl, and a mixture of two or more thereof, preferably from the group consisting of C1-C3-alcohols, C1-C3-alkyl nitriles, dialkyl ketones of the formula R₂—CO—R₃, where R₂ and R₃ are each independently selected from the group consisting of C1-C3-alkyl, and a mixture of two or more thereof, more preferably from the group consisting of methanol, acetonitrile, acetone and a mixture thereof. The hydrogen peroxide is preferably admixed in the form of a methanolic or aqueous, preferably aqueous, solution. The hydrogen peroxide content of the preferably aqueous solution is not subject to any special restrictions and preferably ranges from 25 to 75% by weight, more preferably from 40 to 70% by weight, based on the overall weight of the aqueous solution.

The mixture provided as per (i) preferably comprises no strong nonnucleophilic inorganic acid, preferably no sulfuric acid.

Step (ii)

Catalyst

The inventors found that, surprisingly, the catalyst employed in (ii) has a high level of selectivity for the target product 2-alkoxycyclohexanol. The catalyst employed as per (ii) is not subject to any special restrictions. Preferably, however, the zeolitic material of framework structure MWW evinces one or more of the following features as per the itemized embodiments, including the combinations of embodiments as per the stated dependencies:
1. The zeolitic material of framework structure MWW wherein the framework of the zeolitic material comprises boron and titanium, wherein preferably not less than 99% by weight, more preferably not less than 99.5% by weight, more preferably not less than 99.9% by weight of the framework of the zeolitic material consists of silicon, titanium, boron, oxygen and hydrogen.
2. The zeolitic material according to Embodiment 1 wherein the molar ratio of B:Si is in the range from 0.02:1 to 0.5:1, preferably from 0.05:1 to 0.15:1 and the molar ratio of Ti:Si is in the range from 0.01:1 to 0.05:1, preferably from 0.017:1 to 0.025:1.
3. The zeolitic material according to Embodiment 1 or 2 wherein the zeolitic material is present in the calcined state.
4. The zeolitic material according to Embodiment 3 wherein the calcined state of the zeolitic material is obtained by subjecting the zeolitic material in its non-calcined state to a calcination at a temperature in the range from 500 to 700° C., preferably from 550 to 700° C., more preferably from 600 to 700° C., preferably for a time period in the range from 0.1 to 24 h, more preferably from 1 to 18 h, more preferably from 6 to 12 h, preferably in an atmosphere comprising oxygen.
5. The zeolitic material according to any one of Embodiments 1 to 4 with an MWW-templating compound content of not more than 0.5% by weight, preferably of not more than 0.2% by weight, more preferably of not more than 0.1% by weight, based on the overall weight of the zeolite and reckoned as total organic carbon (TOC) content of the calcined zeolitic material.
6. The zeolitic material according to any one of Embodiments 1 to 5 wherein the ²⁹Si NMR spectrum of the zeolitic material comprises:
   a first signal in the range from −95.0 to −105.0 ppm,
   a second signal in the range from −105.0 to −115.0 ppm,
   a third signal in the range from −115.0 to −125.0 ppm,
   wherein the ratio of the integral of the range of the first signal to the integral of the range of the third signal is preferably in the range from 0.6 to 1.1, more preferably in the range from 0.7 to 1.0, more preferably in the range from 0.8 to 0.9.
7. The zeolitic material according to any one of Embodiments 1 to 6 wherein the ¹¹B NMR spectrum of the zeolitic material comprises:
   a first signal in the range from 20.0 to 10.0 ppm,
   a second signal in the range from 10.0 to 1.0 ppm, preferably having a peak in the range from 6.5 to 5.5 ppm, more preferably from 6.2 to 5.8 ppm,
   a third signal in the range from 1.0 to −7.0 ppm, preferably having a peak in the range from −2.4 to −3.4 ppm, more preferably from −2.7 to −3.1 ppm,
   a fourth signal in the range from −7.0 to −16.0 ppm,
   wherein the ratio of the integral of the range of the third signal to the integral of the range of the second signal is preferably in the range from 1.00 to 1.15, more preferably in the range from 1.05 to 1.15, more preferably in the range from 1.10 to 1.15.
8. The zeolitic material according to any one of Embodiments 1 to 7 with a water uptake in the range from 12.0 to 16.0% by weight, preferably from 12.0 to 15% by weight, more preferably from 12 to 14% by weight.
9. The zeolitic material according to any one of Embodiments 1 to 8 with a specific surface area (BET) in the range from 400 to 500 m²/g, preferably from 410 to 490 m²/g, more preferably from 420 to 480 m²/g, as determined to DIN 66131.
10. The zeolitic material according to any one of Embodiments 1 to 9 wherein the infrared spectrum of the zeolitic material comprises a band at (3748±20) cm⁻¹, a band at (3719±20) cm⁻¹, a band at (3689±20) cm⁻¹, a band at (3623±20) cm⁻¹, a band at (3601±20) cm⁻¹ and a band at (3536±20) cm⁻¹.
11. The zeolitic material according to any one of Embodiments 1 to 10 characterized by an x-ray diffractogram having peaks at 2 theta angles of (7.2±0.1)°, (14.5±0.1)°, (22.1±0.1)°, (22.7±0.1)°, (23.0±0.1)°, (24.0±0.1)°, (25.3±0.1)°, (26.3±0.1)°, (27.3±0.1)°, (28.1±0.1)°.
12. The zeolitic material according to Embodiment 11, characterized by an x-ray diffractogram further comprising peaks at 2 theta angles of (7.0±0.1)°, (8.1±0.1)°, (10.1±0.1)°, (14.3±0.1)°, (20.4±0.1)°, (21.9±0.1)°, (28.9±0.1)°, (33.8±0.1)°, (47.0±0.1)°, (65.4±0.1)°, (66.4±0.1)°.
13. The zeolitic material according to any one of Embodiments 1 to 12 with a boron content, reckoned as elemental boron, in the range from 0.9 to 2.2% by weight, preferably from 1.0 to 2.0% by weight, more preferably from 1.1 to 1.8% by weight, more preferably from 1.2 to 1.6% by weight, based on the overall weight of the zeolitic material.
14. The zeolitic material according to any one of Embodiments 1 to 13 with a titanium content, reckoned as elemental titanium, in the range from 0.9 to 3% by weight, preferably from 1.0 to 2.0% by weight, more preferably from 1.1 to 1.8% by weight, more preferably from 1.2 to 1.6% by weight, based on the overall weight of the zeolitic material.
15. The zeolitic material according to any one of Embodiments 1 to 14 with a boron content, reckoned as elemental boron, in the range from 1.2 to 1.5% by weight and a titanium content, reckoned as elemental titanium, in the range from 1.2 to 1.5% by weight, based on the overall weight of the zeolitic material.
16. The zeolitic material according to any one of Embodiments 1 to 15 in a molding.
17. The zeolitic material according to Embodiment 16 wherein the molding further comprises at least one binder material, preferably silicon dioxide.
18. The zeolitic material according to any one of Embodiments 1 to 17, obtainable or obtained as per a method comprising
   (a) providing an aqueous synthesis mixture comprising a silicon source, a boron source, a titanium source and an MWW-templating compound, wherein the temperature of the aqueous synthesis mixture is not more than 50° C.;
   (b) heating the aqueous synthesis mixture provided as per (a) from the temperature of not more than 50° C. to a temperature in the range from 160 to 190° C. in the course of a period of at most 24 h;
   (c) subjecting the synthesis mixture as per (b) to hydrothermal synthesis conditions under autogenous pressure in a closed system at a temperature in the range from 160 to 190° C. to obtain a precursor to the zeolite of framework structure MWW in its mother liquor;
   (d) separating the precursor to the zeolite of framework structure MWW off from its mother liquor;
   (e) calcining the MWW framework structure zeolite precursor separated off as per (d) to obtain the zeolite of framework structure MWW.
19. The zeolitic material according to Embodiment 18 wherein the aqueous synthesis mixture provided as per (a) is obtained by admixing the silicon source to an aqueous mixture comprising the boron source, the titanium source and the MWW-templating compound.
20. The zeolitic material according to Embodiment 18 or 19 wherein the aqueous mixture comprising the boron source, the titanium source and the MWW-templating compound is obtained by admixing a mixture comprising the silicon source and some of the MWW-templating compound to an aqueous mixture comprising the boron source and some of the MWW-templating compound, wherein the mixture comprising some of the MWW-templating compound and some of the titanium source preferably comprises no water.
21. The zeolitic material according to any one of Embodiments 18 to 20 wherein the aqueous synthesis mixture after the step of admixing the silicon source is stirred at a temperature of not more than 50° C. for a time period in the range from 45 to 180 min, preferably from 60 to 120 min, more preferably from 80 to 100 min.
22. The zeolitic material according to any one of Embodiments 18 to 21 wherein as per (a) the silicon source is selected from the group consisting of fumed silica, colloidal silica, silicon alkoxides and a mixture of two or more thereof, preferably from the group consisting of fumed silica, colloidal silica and a mixture thereof, wherein the silicon source is more preferably fumed silica;
   the boron source is selected from the group consisting of boric acid, borates, boron oxide and a mixture of two or more thereof, preferably from the group consisting of boric acid, borates and a mixture thereof, wherein the boron source is more preferably boric acid;
   the titanium source is selected from the group consisting of titanium alkoxides, titanium halides, titanium salts, titanium dioxide and a mixture of two or more thereof, preferably from the group consisting of titanium alkoxides, titanium halides and a mixture thereof, wherein the titanium source is more preferably a titanium alkoxide, more preferably titanium tetrabutoxide;
   the MWW-templating compound is selected from the group consisting of piperidine, hexamethyleneimine, N,N,N,N′,N′,N′-hexamethyl-1,5-pentanediammonium salts, 1,4-bis(N-methylpyrrolidinyl)butane, octyltrimethylammonium hydroxide, heptyltrimethyl-ammonium hydroxide, hexyltrimethylammonium hydroxide and a mixture of two or more thereof, preferably from the group consisting of piperidine, hexamethyleneimine and a mixture thereof, wherein the MWW-templating compound is more preferably piperidine.
23. The zeolitic material according to any one of Embodiments 18 to 22 wherein the aqueous synthesis mixture comprises:
   the boron source, reckoned as elemental boron, based on the silicon source, reckoned as elemental silicon, in a molar ratio ranging from 0.18:1 to 5.2:1, preferably from 0.5:1 to 3:1;
   the titanium source, reckoned as elemental titanium, based on the silicon source, reckoned as elemental silicon, in a molar ratio ranging from 0.005:1 to 0.15:1, preferably from 0.01:1 to 0.1:1;
   the MWW-templating compound based on the silicon source, reckoned as elemental silicon, in a molar ratio ranging from 0.4:1 to 4.2:1, preferably from 0.6:1 to 2:1; the water, based on the silicon source, reckoned as elemental silicon, in a molar ratio ranging from 1:1 to 30:1, preferably from 2:1 to 25:1.
24. The zeolitic material according to any one of Embodiments 18 to 23 wherein the aqueous synthesis mixture provided as per (a) has a pH in the range from 10 to 13, preferably from 10.5 to 12.5, more preferably from 11 to 12, as determined using a pH-sensitive glass electrode.
25. The zeolitic material according to any one of Embodiments 18 to 24 wherein step (b) of heating the aqueous synthesis mixture provided as per (a) from the temperature of not more than 50° C. to a temperature in the range from 160 to 190° C. is effected within a time period ranging from 2 to 18 h, preferably from 4 to 14 h, more preferably from 8 to 12 h, preferably under agitation.
26. The zeolitic material according to any one of Embodiments 18 to 25 wherein step (b) of heating the mixture provided as per (a) from the temperature of not more than 50° C. to a temperature in the range from 160 to 190° C. is carried out in a continuous manner.
27. The zeolitic material according to any one of Embodiments 18 to 26 wherein step (c) of subjecting the synthesis mixture to hydrothermal synthesis conditions under autogenous pressure is effected for a time period in the range from 80 to 200 h, preferably from 100 to 180 h, more preferably from 120 to 160 h, preferably under at least temporary agitation.
28. The zeolitic material according to any one of Embodiments 18 to 27 wherein step (d) of separating off comprises
   (d.1) washing the precursor to the zeolitic material of framework structure MWW, preferably with water, until the water used for said washing has a pH of less than 10, as determined using a pH-sensitive glass electrode;
   (d.2) drying the washed precursor to the zeolitic material of framework structure

MWW, preferably at a temperature in the range from 10 to 150° C., more preferably at a temperature in the range from 30 to 130° C., preferably in an atmosphere comprising oxygen.
29. The zeolitic material according to any one of Embodiments 18 to 28 wherein the precursor to the zeolitic material of framework structure MWW is treated neither before nor during (d) with an aqueous solution having a pH of not more than 6, as determined using a pH-sensitive glass electrode.
30. The zeolitic material according to any one of Embodiments 18 to 29 wherein step (e) of calcining is carried out at a temperature in the range from 500 to 700° C., preferably from 550 to 700° C., more preferably from 600 to 700° C., preferably for a time period in the range from 0.1 to 24 h, more preferably from 1 to 18 h, more preferably from 6 to 12 h.
31. The zeolitic material according to any one of Embodiments 18 to 30 wherein the MWW framework structure zeolitic material precursor obtained from (c) is not treated before (d) with an aqueous solution having a pH of not more than 6, as determined using a pH-sensitive glass electrode, and wherein the calcined zeolitic material of framework structure MWW is not treated after (d) with an aqueous solution having a pH of not more than 6, as determined using a pH-sensitive glass electrode.
32. The zeolitic material according to any one of Embodiments 18 to 31 wherein the boron content of the precursor to the MWW framework structure zeolitic material which is subjected to a calcination is not less than 90%, preferably not less than 95%, more preferably not less than 98% of the boron content of the precursor obtained from the hydrothermal synthesis as per (c).
33. The zeolitic material according to any one of Embodiments 18 to 32 wherein the boron content of the MWW framework structure zeolitic material obtained from the calcining step as per (d) is not less than 90%, preferably not less than 95%, more preferably not less than 98% of the boron content of the precursor obtained from the hydrothermal synthesis as per (c).
34. The zeolitic material according to any one of Embodiments 18 to 33 wherein the method further comprises
   (f) molding the MWW framework structure zeolitic material obtained from (d) or (e) to obtain a molding;
   (g) optionally drying and/or calcining the molding obtained from (f).
35. The zeolitic material according to any one of Embodiments 18 to 34 wherein the MWW framework structure zeolitic material is not before (f) treated with an aqueous solution having a pH of not more than 6, as determined using a pH-sensitive glass electrode, and wherein the molding comprising the zeolitic material of framework structure MWW is not after steps (f) and (g) treated with an aqueous solution having a pH of not more than 6, as determined using a pH-sensitive glass electrode.
36. The zeolitic material according to Embodiment 34 or 35 wherein the boron content of the MWW framework structure zeolitic material in the molding obtained as per (f) or (g), preferably as per (g), is not less than 90%, preferably not less than 95%, more preferably not less than 98% of the boron content of the precursor obtained from the hydrothermal synthesis as per (c).
37. A molding comprising a zeolitic material according to any one of Embodiments 1 to 15 or 18 to 33.

Reaction as Per Step (ii)

As far as the amount of catalyst is concerned, the mass ratio of hydrogen peroxide:zeolitic material of framework structure MWW is preferably in the range from 10:1 to 0.1:1, preferably from 1:1 to 0.2:1, more preferably from 0.75:1 to 0.25:1, at the start of the reaction as per (ii).

The reaction as per (ii) may generally be carried out as per any suitable procedure. Options thus include, for example, a discontinuous process in one or more batch reactors or a continuous process in one or more reactors operated in a continuous manner and optionally interconnected in series and/or in parallel.

The procedure for performing the reaction as per (ii) in a discontinuous process is not subject to any special restriction. A suitable reactor for the reaction as per (ii) is, for example, a reactor fitted with suitable heating means, a suitable stirrer and a reflux condenser. The reaction as per (ii) is preferably carried out in an open system. The reaction as per (ii) is preferably carried out under suitable agitation of the reaction mixture, for example stirring, in which case the energy input due to agitation may be varied or kept substantially constant during the reaction. The energy input may be suitably chosen according to, for example, the volume of the reaction mixture, the form of the catalyst or the reaction temperature. When the reaction as per (ii) is carried out in batch mode, the catalyst used as per (ii) is preferably an MWW framework structure zeolitic material as described above in Embodiments 1 to 15 and 18 to 33.

The procedure for performing the reaction as per (ii) in a continuous process is not subject to any special restriction. The continuous process is preferably carried out using, for example, a fixed bed catalyst, in which case the catalyst used as per (ii) is preferably a molding as described above in Embodiments 16, 17 and 34 to 36, comprising the zeolitic material of framework structure MWW and preferably one or more than one binder material, preferably silicon dioxide. Catalyst velocity in the continuous process is preferably in the range from 0.05 to 5 mol/kg/h, more preferably from 0.1 to 4 mol/kg/h, more preferably from 0.2 to 3 mol/kg/h, catalyst velocity being defined as mol (of hydrogen peroxide)/kg (of MWW framework structure zeolitic material)/h. For a continuous process, the mixture as per (i) is preferably provided as a liquid stream which is routed into the one or more reactors and subjected therein to reaction conditions as per (ii). For the continuous process, it is also possible to route the individual components of the mixture as per (i) in the form of two or more streams, which may comprise the individual components or a mixture thereof, into the one or more reactors where the individual streams are combined after reactor entry to form the mixture as per (i). Two or more reactors operated in a continuous manner may be interconnected two or more at a time in parallel and/or two or more at a time in series. Between two reactors interconnected in series there may be provided one or more interstages, for example to intermediately recover product of value. Between two reactors interconnected in series there may further be supplied one or more of the starting materials cyclohexene, R₁OH alcohol, hydrogen peroxide and optional solvent.

Irrespective of the type of processing mode chosen, the reaction as per (ii) is performable using one or more, mutually different catalysts comprising a zeolitic material of framework structure MWW and comprising B and Ti in the framework. The catalysts may differ, for example, with regard to the chemical composition or the manner of making the zeolitic material of framework structure MWW. When moldings are used, the catalysts may further differ for example with regard to the properties of the molding, e.g., in the geometry of the molding, the porosity of the molding, the binder content of the molding, the binder material or the percentage content of MWW framework structure zeolitic material. The reaction as per (ii) is preferably carried out in the presence of a single catalyst of the present invention.

Following the reaction as per (ii), the catalyst used is separated off from the mixture comprising the compound of formula (I). When the reaction is carried out in a continuous manner, for example in a fixed bed reactor, there is no need for a step to separate off the catalyst, since the reaction mixture leaves the reactor and the catalyst remains behind in the fixed bed reactor. When the reaction is carried out in discontinuous mode, for example in a batch reactor, the catalyst, which is preferably employed in the form of a powder, is removable using a suitable method of separation, examples being filtration, ultrafiltration, diafiltration, centrifugation and/or decanting.

Following removal, the removed catalyst may be subjected to one or more washing steps with one or more suitable washing liquids. Useful washing liquids include, for example, water, ethers such as dioxane, for example 1,4-dioxane, alcohols such as, for example, methanol, ethanol, propanol or a mixture of two or more thereof. Dioxanes are preferred for use as washing liquid. The washing step is preferably carried out at a temperature in the range from 10 to 50° C., more preferably at a temperature in the range from 15 to 40° C., more preferably at a temperature in the range from 20 to 30° C.

When the conversion rate, the selectivity or both the conversion rate and the selectivity offered by the catalyst according to the present invention decrease to below certain values, the catalyst can be regenerated in a suitable manner, for example by washing with one or more suitable washing media, or by drying in one or more suitable atmospheres, at one or more suitable temperatures and at one or more suitable pressures, or by calcination in one or more suitable atmospheres, at one or more suitable temperatures and at one or more suitable pressures, or by a combination of two or more of these measures, which may each be carried out one or more times for within one or more suitable time periods.

The reaction as per (ii) is preferably carried out at a reaction mixture temperature in the range from 40 to 150° C., more preferably at a reaction mixture temperature in the range from 50 to 125° C., more preferably at a reaction mixture temperature in the range from 55 to 100° C. When the reaction as per (ii) is carried out in a discontinuous manner, for example as a batch reaction, it is preferably carried out at the boiling point of the liquid mixture, more preferably under reflux.

When the reaction as per (ii) is carried out in a discontinuous manner, for example as a batch reaction, the duration of the reaction is preferably in the range from 1 to 12 h, more preferably in the range from 1.5 to 10 h, more preferably in the range from 2 to 8 h.

When the reaction as per (ii) is carried out in a discontinuous manner, the term “at the start of the reaction” relates generally to the point in time at which all the starting materials, including the catalyst, are simultaneously present in the reaction mixture and, depending on the temperature, the reaction of the cyclohexane starts. When the reaction as per (ii) is carried out in a continuous manner, the term “at the start of the reaction” relates generally to the point in time at which the mixture provided as per (i) comes into contact with the catalyst.

Preferably, the mole percentage for the compound of formula (I) in the mixture obtained from the reaction as per (ii), based on the sum total of mole percentages for the compounds of formulae (I), (II), (III), (IV) and (V)

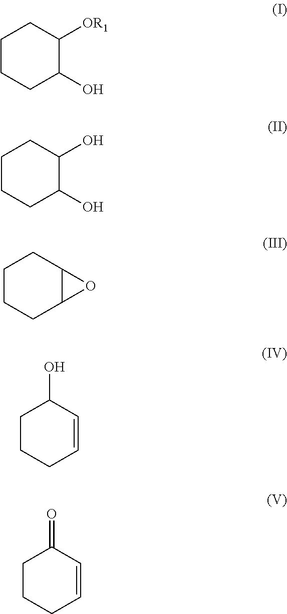

in the mixture obtained from the reaction as per (ii) is not less than 85%, preferably not less than 90%. It is not a mandatory requirement here that every one of compounds (II), (III), (IV) and (V) be present in the mixture obtained as per (ii); on the contrary, the mixture obtained as per (ii) may comprise just one, just two, just three or all four of the compounds (II), (III), (IV) and (V) in addition to the compound (I).

The mixture obtained as per (ii), comprising the compound of formula (I), is preferably worked up to recover the compound of formula (I). When the conversion of hydrogen peroxide during the reaction is incomplete, it is preferable to precede the workup by removing the unconverted hydrogen peroxide present in the mixture, for example by decomposing it through admixture of suitable substances, for example via quenching. Examples of substances suitable for decomposing the excess hydrogen peroxide include, for example, tertiary amines, polyamines, salts of heavy metals such as iron, manganese, cobalt and vanadium, sulfinic acids, mercaptans, dithionites, sulfites and strong acids and bases. The decomposition of the excess hydrogen peroxide is preferably carried out using an alkali or alkaline earth metal sulfite, more preferably alkali metal sulfite, more preferably sodium sulfite.

The mixture obtained as per (ii), comprising the compound of formula (II), is preferably worked up by
(iii) separating the compound of formula (II) off from the mixture obtained from the reaction as per (ii) to obtain a mixture depleted with respect to the compound of formula (II).

The separating step as per (iii) is carried out with preference when the mole percentage for the compound of formula (II) in the mixture obtained as per (ii), comprising the compounds of formulae (I) and (II) and optionally at least one of the compounds as per formulae (III), (IV) and (V)

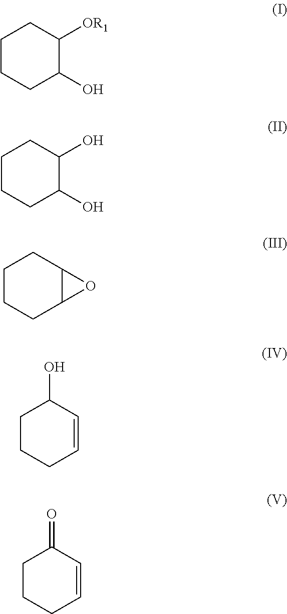

based on the sum total of the molar percentages for the compounds of formulae (II), (III), (IV) and (V) in the mixture is not less than 70%, more preferably not less than 75%, more preferably not less than 80%.

Preferably, the mole percentage for the compound of formula (I) in the mixture obtained as per (iii), depleted with respect to the compound of formula (II), based on the sum total of the mole percentages for the compounds of formulae (I), (II), (III), (IV) and (V) in the mixture, is not less than 90%, preferably not less than 95%.

The separating step as per (iii) is in principle not subject to any restriction and is performable using any suitable separation procedure such as, for example, distillation, sublimation, chromatography, crystallization or a combination thereof.

A further possible workup comprises the step of
(iv) separating the compound of formula (I) off from the mixture obtained from the reaction as per (ii) or from the mixture obtained from the separating step as per (iii) as being depleted with respect to the compound of formula (II).

When the compound of formula (I) is separated off from the mixture obtained from the reaction as per (ii), the compound of formula (II) may subsequently be separated off from the resulting mixture.

The separating step as per (iv) is in principle not subject to any restriction and is performable using any suitable separation procedure such as, for example, distillation, sublimation, chromatography, crystallization or a combination thereof. Preferably distillation is used to separate the compound of formula (I) off from the mixture obtained from (ii) and (iii), the distillation not being in principle subject to any restriction. It is preferable for the separation as per (iv) to be carried out by distillation at a pot temperature of 25 to 200° C., more preferably 30 to 150° C. and a pressure of 5 to 1500 mbar, more preferably 10 to 1300 mbar.

The mixture obtained by the separating step as per (iv), concentrated with respect to the compound of formula (I), comprises the compound of formula (I) at preferably not less than 90% by weight and more preferably at not less than 95% by weight.

The present invention accordingly provides with preference a method of preparing a compound of formula (I)

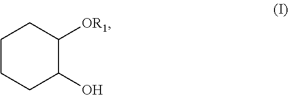

where R₁ is methyl, comprising
(i) providing a liquid mixture comprising cyclohexene, methanol and hydrogen peroxide in a molar cyclohexene:methanol ratio ranging from 1:3 to 1:30 and in a molar cyclohexene:hydrogen peroxide ratio ranging from 1.5:1 to 4.5:1,
(ii) reacting the cyclohexene with the hydrogen peroxide and the methanol in the mixture provided as per (i) in the presence of a catalyst comprising a zeolite of framework structure MWW at a temperature in the range from 55 to 100° C. to obtain a mixture comprising the compound of formula (I), wherein the framework of the zeolite as per (ii) comprises silicon, titanium, boron, oxygen and hydrogen and the zeolite has a boron content, reckoned as elemental boron, in the range from 0.9% to 2.2% by weight and a titanium content, reckoned as elemental titanium, in the range from 0.9% to 3.0% by weight, based on the overall weight of the zeolite;
(iii) separating the compound of formula (II)

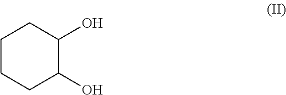

off from the mixture obtained from the reaction as per (ii) to obtain a mixture depleted in respect of the compound of formula (II);
(iv) optionally separating the compound of formula (I) off from the mixture obtained from the separating step as per (iii), depleted in respect of the compound of formula (II).

Mixture Comprising the Compound of Formula (I) and Use Thereof

The present invention also provides a mixture comprising a compound of formula (I)

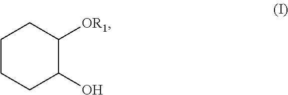

where R₁ is alkyl of 1 to 4 carbon atoms, obtainable or obtained as per that method of the present invention wherein the mole percentage for the compound of formula (I) in the mixture, based on the sum total of mole percentages for the compounds of formulae (I), (II), (III), (IV) and (V)

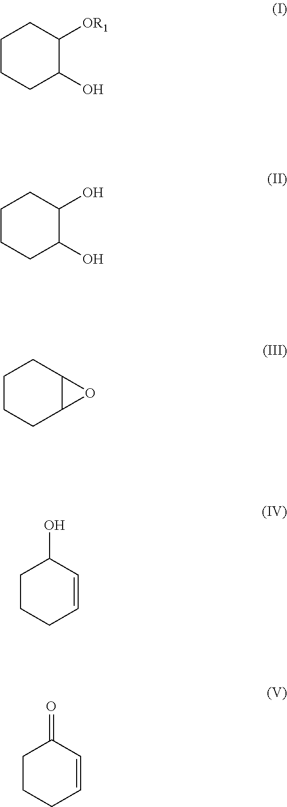

in the mixture is not less than 85%, preferably not less than 90%.

The mixture of the present invention is preferably depleted of the compound of formula (II) by the above-described separating step (iii).

The present invention further relates to a method of using the mixture of the present invention, comprising a compound of formula (I), in the manufacture of a compound of formula (VI)

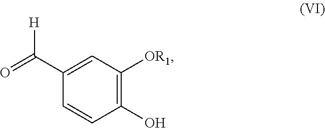

where R₁ is alkyl of 1 to 4 carbon atoms, as defined above.

The use referred to is preferable in a method of preparing a compound of formula (VI) comprising the steps of:
(A) dehydrogenating the formula (I) compound in the mixture of the present invention to obtain a mixture comprising a compound of formula (II)

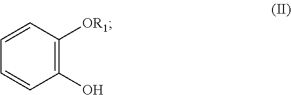

(B) formylating the formula (II) compound in the mixture obtained as per (A) to obtain a mixture comprising the compound of formula (IV).

The present invention is further illustrated by the following embodiments and combinations of embodiments apparent from dependencies and other references:
1. A method of preparing a compound of formula (I)

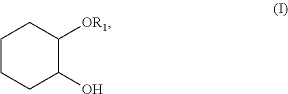

where R₁ is alkyl of 1 to 4 carbon atoms, comprising
(i) providing a liquid mixture comprising cyclohexene, an alcohol R₁OH, hydrogen peroxide and optionally a solvent;
(ii) reacting the cyclohexene with the hydrogen peroxide and the alcohol R₁OH in the mixture provided as per (i) in the presence of a catalyst comprising a zeolite of framework structure MWW to obtain a mixture comprising the compound of formula (I)
where the framework of the zeolite as per (ii) comprises silicon, titanium, boron, oxygen and hydrogen.
2. The method according to Embodiment 1 wherein R₁ is methyl or ethyl, preferably methyl.
3. The method according to Embodiment 1 or 2 wherein the liquid mixture provided as per (i) has a molar ratio prior to the reaction as per (ii) of cyclohexene:R₁OH in the range from 1:1 to 1:50, preferably from 1:3 to 1:30, more preferably from 1:5 to 1:10.
4. The method according to any one of Embodiments 1 to 3 wherein the liquid mixture provided as per (i) has a molar ratio prior to the reaction as per (ii) of cyclohexene:hydrogen peroxide in the range from 1:1 to 5:1, preferably from 1.5:1 to 4.5:1, more preferably from 2:1 to 4:1.
5. The method according to any one of Embodiments 1 to 4 wherein the liquid mixture provided as per (i) comprises a solvent, preferably selected from the group consisting of C1-C6-alkyl nitriles, dialkyl ketones of the formula R₂—CO—R₃, where R₂ and R₃ are each independently selected from the group consisting of C1-C6-alkyl, and a mixture of two or more thereof, more preferably from the group consisting of C1-C3-alkyl nitriles, dialkyl ketones of the formula R₂—CO—R₃, where R₂ and R₃ are each independently selected from the group consisting of C1-C3-alkyl, and a mixture of two or more thereof, more preferably from the group consisting of acetonitrile, acetone and a mixture thereof.
6. The method according to Embodiment 5 wherein the molar ratio of solvent:cyclohexene in the liquid mixture provided as per (i) is in the range from 20:1 to 1:1, preferably from 15:1 to 1:1, more preferably from 10:1 to 1:1, before the reaction as per (ii) subject to the proviso that when the solvent in the mixture comprises a mixture of two or more solvents, the molar ratio of solvent:cyclohexene is based on the mixture of solvents.
7. The method according to any one of Embodiments 1 to 4 wherein the mixture provided as per (i) comprises no solvent.
8. The method according to any one of Embodiments 1 to 7 wherein the mixture as per (i) is provided by admixing hydrogen peroxide to a mixture comprising the cyclohexene, the alcohol R₁OH and optionally the solvent.
9. The method according to Embodiment 8 wherein the hydrogen peroxide is admixed in the form of an aqueous solution, wherein the hydrogen peroxide content of the aqueous solution is preferably in the range from 25 to 75% by weight, more preferably from 40 to 70% by weight, based on the overall weight of the aqueous solution.
10. The method according to any one of Embodiments 1 to 9 wherein the reaction as per (ii) is carried out at a temperature in the range from 40 to 150° C., preferably from 50 to 125° C., more preferably from 55 to 100° C.
11. The method according to any one of Embodiments 1 to 10 wherein the reaction as per (ii) is carried out at the boiling point of the liquid mixture, preferably under reflux.
12. The method according to any one of Embodiments 1 to 11 wherein the duration of the reaction as per (ii) is in the range from 1 to 12 h, preferably from 1.5 to 10 h, more preferably from 2 to 8 h.
13. The method according to any one of Embodiments 1 to 12 wherein the mass ratio of hydrogen peroxide: zeolite of framework structure MWW at the start of the reaction as per (ii) is in the range from 10:1 to 0.1:1, preferably from 1;1 to 0.2:1, more preferably from 0.75:1 to 0.25:1.
14. The method according to any one of Embodiments 1 to 13 wherein not less than 99% by weight, preferably not less than 99.5% by weight, more preferably not less than 99.9% by weight of the framework of the zeolite as per (ii) consists of silicon, titanium, boron, oxygen and hydrogen.
15. The method according to any one of Embodiments 1 to 14 wherein, in the zeolite of framework structure MWW, the molar ratio of B:Si is in the range from 0.02:1 to 0.5:1, preferably from 0.05:1 to 0.15:1 and the molar ratio of Ti:Si is in the range from 0.01:1 to 0.05:1, preferably from 0.017:1 to 0.025:1.
16. The method according to any one of Embodiments 1 to 15 wherein the zeolite of framework structure MWW is obtainable or obtained as per a method comprising
   (a) providing an aqueous synthesis mixture comprising a silicon source, a boron source, a titanium source and an MWW-templating compound, wherein the temperature of the aqueous synthesis mixture is not more than 50° C.;
   (b) heating the aqueous synthesis mixture provided as per (a) from the temperature of not more than 50° C. to a temperature in the range from 160 to 190° C. in the course of a period of at most 24 h;
   (c) subjecting the synthesis mixture as per (b) to hydrothermal synthesis conditions under autogenous pressure in a closed system at a temperature in the range from 160 to 190° C. to obtain a precursor to the zeolite of framework structure MWW in its mother liquor;
   (d) separating the precursor to the zeolite of framework structure MWW off from its mother liquor;
   (e) calcining the MWW framework structure zeolite precursor separated off as per (d) to obtain the zeolite of framework structure MWW.
17. The method according to any one of Embodiments 1 to 16 wherein the catalyst, preferably in the form of a molding, comprises a binder, preferably a silica binder, in addition to the MWW framework structure zeolite.
18. The method according to any one of Embodiments 1 to 17 wherein the mixture provided as per (i) comprises no sulfuric acid, preferably no strong nonnucleophilic acid.
19. The method according to any one of Embodiments 1 to 18 wherein the mole percentage for the compound of formula (I) in the mixture obtained from the reaction as per (ii), based on the sum total of mole percentages for the compounds of formulae (I), (II), (III), (IV) and (V)

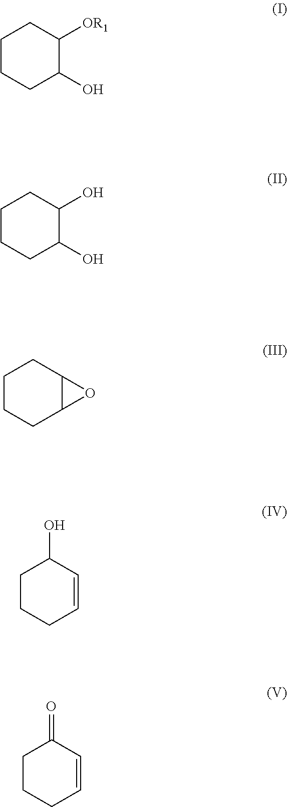

in the mixture obtained from the reaction as per (ii) is not less than 85%, preferably not less than 90%.
20. The method according to any one of Embodiments 1 to 19 wherein the mixture obtained from the reaction as per (ii) comprises the compounds of formulae (I) and (II) and optionally at least one of the compounds as per formulae (III), (IV) and (V)

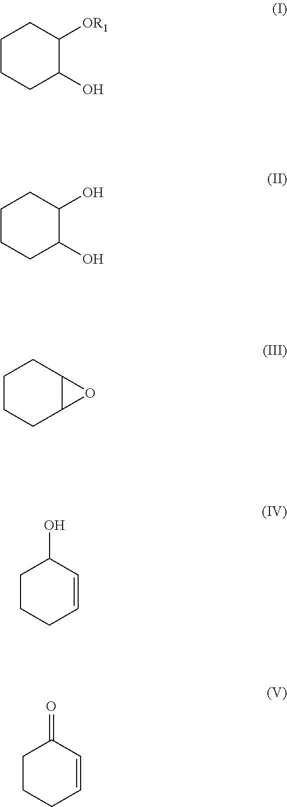

and wherein the mole percentage for the compound of formula (II) in the mixture, based on the sum total of mole percentages for the compounds of formulae (II), (III), (IV) and (V) in the mixture is preferably not less than 70%, more preferably not less than 75% and more preferably not less than 80%, comprising
   (iii) separating the compound of formula (II) off from the mixture obtained from the reaction as per (ii) to obtain a mixture depleted with respect to the compound of formula (II).
21. The method according to any one of Embodiments 1 to 20, comprising
   (iv) separating the compound of formula (I) off from the mixture obtained from the reaction as per (ii) or from the mixture obtained from the separating step as per (iii) as being depleted with respect to the compound of formula (II).
22. A mixture comprising a compound of formula (I)

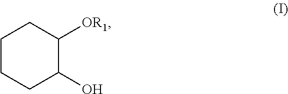

where R₁ is alkyl of 1 to 4 carbon atoms, obtainable or obtained as per a method according to any one of Embodiments 1 to 20 wherein the mole percentage for the compound of formula (I) in the mixture, based on the sum total of mole percentages for the compounds of formulae (I), (II), (III), (IV) and (V)

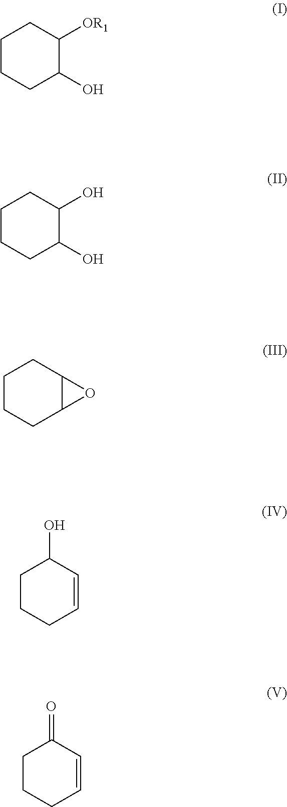

in the mixture is not less than 85%, preferably not less than 90%.
23. The method of using a mixture according to Embodiment 22 in the manufacture of a compound of formula (VI)

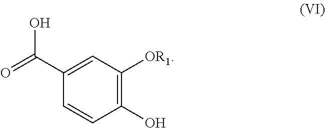

The invention is more particularly elucidated in the reference, inventive and comparative examples which follow.

### EXAMPLES

The experimental examples which follow utilized the following starting materials:

1. piperidine (Sigma-Aldrich)

2. boric acid (Bernd Kraft)

3. tetrabutyl orthotitanate (Alfa Aesar)

4. CAB-O-SIL® M7D and CAB-O-SIL® M5 (Cabot) fumed silica

5. Ludox® AS-40 (Sigma-Aldrich) colloidal silica

6. cyclohexene (Sigma Aldrich)

7. methanol (Fluka)

8. hydrogen peroxide (Solvay)

### Reference Example 1

### Measurement of ¹¹B Solid State NMR Spectra

The ¹¹B solid state NMR experiments were carried out using a Bruker Avance III spectrometer operating at 400 MHz ¹H Larmor frequency (Bruker Biospin, Germany). The samples were stored at room temperature and 63% relative humidity before being packed into 4 mm ZrO₂ rotors. Measurements were performed under 10 kHz magic angle spinning at room temperature. ¹¹B-Spectra were obtained using ^{11B} 15° pulse excitation of 1 microsecond (μs) pulse width, an ¹¹B carrier frequency corresponding to −4 ppm in the referenced spectrum, and a scan recycle delay of 1 s. Signal was acquired for 10 ms and accumulated with 5000 scans. Spectra were processed using a Bruker Topspin with 30 Hz exponential line broadening, phasing and baseline correction across the full width of the spectrum. Spectra were indirectly referenced to 1% TMS in CDCl₃ on the unified chemical shift scale according to IUPAC (Pure Appl. Chem., vol. 80, No. 1, p. 59) using glycine with carbonyl peak at 175.67 ppm as secondary standard.

### Reference Example 2

### Measurement of ²⁹Si Solid State NMR Spectra

²⁹Si solid state NMR experiments were carried out using a Bruker Advance III spectrometer operating at 400 MHz ¹H Larmor frequency (Bruker Biospin, Germany). The samples were stored at room temperature and 63% relative humidity before being packed into 4 mm ZrO₂ rotors. Measurements were performed under 10 kHz magic angle spinning at room temperature. ²⁹Si -Spectra were obtained using ²⁹Si 90° pulse excitation of 5 microsecond (μs) pulse width, a ²⁹Si carrier frequency corresponding to −112 ppm in the referenced spectrum, and a scan recycle delay of 120 s. Signal was acquired for 20 milliseconds (ms) at 63 kHz high-power proton decoupling and accumulated for at least 16 hours. Spectra were processed using a Bruker Topspin with 50 Hz exponential line broadening, phasing and baseline correction across the full width of the spectrum. Spectra were indirectly referenced to 1% TMS in CDCl₃ on the unified chemical shift scale according to IUPAC (Pure Appl. Chem., vol. 80, No. 1, p. 59) using glycine with carbonyl peak at 175.67 ppm as secondary standard.

### Reference Example 3

### Determination of Water Uptake

Water adsorption/desorption isoterms were performed on a VTI SA instrument from TA Instruments following a step-isotherm program. The experiment consisted in one or more runs performed on a sample material placed on the microbalance pan inside the instrument. Before measurement was started, the residual moisture content of the sample was removed by heating the sample to 100° C. (5 K/min heat ramp) and maintaining the sample under a nitrogen stream for 6 h. After the drying program, the temperature in the cell was lowered to 25° C. and kept isothermal during measurement. The microbalance was calibrated, and the weight of the dried sample was balanced (0.01% by weight maximum deviation in mass). Water uptake by the sample was measured as the increased weight over the dry sample. An adsorption curve was measured first by increasing the relative humidity (expressed as % by weight of water in the atmosphere of the cell) to which the sample was exposed, and measuring the water uptake of the sample as the equalizing weight. The relative humidity was increased from 5% by weight to 85% by weight in 10% by weight increments, with the system policing the relative humidity for each increment, and monitoring the sample weight until attainment of equilibrium conditions after the sample was exposed to 85% by weight to 5% by weight relative humidity in increments of 10% by weight and the change in the weight of the sample (the water uptake) had been monitored and recorded.

### Reference Example 4

### Measurement of Infrared Spectra

FT-IR (Fourier transform infrared) measurements were performed on a Nicolet 6700 spectrometer. The powdered material was compressed into a self-supporting pellet without the use of any additives. The pellet was introduced into a high vacuum cell (HV) accommodated in the FT-1R instrument. Measurement of the sample was preceded by preheating in high vacuum (10⁻⁵ mbar) at 300° C. for 3 h. Spectra were recorded after cooling the cell back down to 50° C. Spectra were recorded in the range from 4000 to 800 cm⁻¹ at a resolution of 2 cm⁻¹. The spectra obtained were depicted in a diagram having the wavelength (cm⁻¹) on the x-axis and the absorption (in arbitrary units “a.u.”). A baseline correction was carried out to quantitatively determine the peak heights and the ratios between these peaks. Changes in the range from 3000-3900 cm⁻¹ were analyzed and the band at 1880±5 cm⁻¹ was used as reference to compare two or more samples.

### Reference Example 5

### Measurement of X-Ray Diffraction Spectra

The x-ray diffraction spectrum was recorded using a D8 Advance Series 2 from Bruker/AXS, which was equipped with a multiple sample changer.

### Example 1

### Preparing an MWW Framework Structure Zeolite Comprising Boron and Titanium as Per the Method of the Preferred Embodiment According to the Present Invention

Deionized water (841.82 g) in a glass beaker was admixed with piperidine (200 g), and the resulting mixture was stirred at room temperature for 5 min. Boric acid (203.8 g) was then admixed to the mixture and dissolved for 20 min, followed by a solution of tetrabutyl orthotitanate (17.75 g) dissolved in piperidine (99.24 g) admixed under agitation at a stirrer speed of 70 rpm, and the resulting mixture was stirred at room temperature for 30 min. The mixture was admixed with fumed silica (Cab-O-Sil® M7D, 147.9 g) under agitation, and the resulting mixture was stirred at room temperature for 1.5 h. The mixture had a pH of 11.3.

The mixture was transferred into a 2.5 I autoclave and slowly heated to 170° C. over 10 hours at a heating rate of about 0.2 K/min and then was maintained at 170° C. for 160 h under agitation at a stirrer speed of 100 rpm. The pressure during the reaction was in the range from 8.3 to 9 bar. The suspension obtained had a pH of 11.2. The suspension was filtered and the filtercake was washed with deionized water until the wash liquor had a pH of less than 10. The filtercake was placed in a drying oven and dried at 120° C. for 48 h, heated to a temperature of 650° C. at a heating rate of 2 K/min and calcined at 650° C. in an air atmosphere for 10 h to obtain a colorless powder (101.3 g). The powder had a boron content of 1.3% by weight, reckoned as elemental boron, a titanium content of 1.3% by weight, reckoned as elemental titanium, and a silicon content of 40% by weight, reckoned as elemental silicon. The hydrocarbon content totaled 0.1% by weight. The water uptake determined as per Reference Example 3 was 13.7% by weight. The ¹¹B solid state NMR spectrum of the zeolitic material is shown in FIG. 1. The ²⁹Si solid state NMR spectrum of the zeolitic material is shown in FIG. 2. The FT-IR spectrum of the zeolitic material is shown in FIG. 3. The x-ray diffraction spectrum of the zeolitic material is shown in FIG. 4. The x-ray diffraction spectrum of the zeolitic material further has the following characteristics:

| Angle | d-Value | Intensity | Intensity |
|---|---|---|---|
| 2-Theta ° | Angstrom | Cps | % |
| 7.013 | 12.59532 | 274 | 20.3 |
| 7.238 | 12.20307 | 468 | 34.7 |
| 8.058 | 10.96352 | 326 | 24.2 |
| 10.137 | 8.71933 | 362 | 26.9 |
| 13.007 | 6.80071 | 134 | 10 |
| 14.278 | 6.19805 | 313 | 23.2 |
| 14.476 | 6.11373 | 408 | 30.3 |
| 14.924 | 5.93142 | 209 | 15.5 |
| 16.143 | 5.48597 | 249 | 18.5 |
| 18.048 | 4.91111 | 119 | 8.8 |
| 19.22 | 4.61418 | 192 | 14.3 |
| 20.448 | 4.33988 | 313 | 23.3 |
| 21.418 | 4.14544 | 239 | 17.8 |
| 21.857 | 4.06307 | 403 | 29.9 |
| 22.142 | 4.01139 | 424 | 31.5 |
| 22.687 | 3.91637 | 413 | 30.7 |
| 22.974 | 3.8681 | 747 | 55.5 |
| 23.97 | 3.70954 | 539 | 40 |
| 25.277 | 3.5206 | 446 | 33.1 |
| 26.321 | 3.38327 | 1346 | 100 |
| 27.289 | 3.26537 | 498 | 37 |
| 28.094 | 3.17361 | 511 | 37.9 |
| 28.947 | 3.08203 | 357 | 26.5 |
| 30.059 | 2.97051 | 210 | 15.6 |
| 32.004 | 2.79431 | 217 | 16.1 |
| 32.669 | 2.73889 | 229 | 17 |
| 33.757 | 2.65307 | 319 | 23.7 |
| 34.829 | 2.57383 | 233 | 17.3 |
| 36.837 | 2.43798 | 209 | 15.5 |
| 37.535 | 2.39427 | 197 | 14.6 |
| 38.316 | 2.34724 | 240 | 17.8 |
| 41.141 | 2.19233 | 204 | 15.2 |
| 41.982 | 2.15035 | 205 | 15.2 |
| 43.358 | 2.08522 | 213 | 15.8 |
| 45.169 | 2.00577 | 255 | 19 |
| 46.654 | 1.94533 | 266 | 19.8 |
| 46.968 | 1.93304 | 284 | 21.1 |
| 48.908 | 1.86079 | 255 | 19 |
| 49.494 | 1.84015 | 259 | 19.2 |
| 49.992 | 1.82295 | 232 | 17.2 |
| 51.38 | 1.77694 | 246 | 18.3 |
| 52.019 | 1.75659 | 255 | 18.9 |
| 53.699 | 1.70552 | 240 | 17.8 |
| 54.686 | 1.67707 | 230 | 17.1 |
| 57.016 | 1.61393 | 233 | 17.3 |
| 57.764 | 1.5948 | 225 | 16.7 |
| 58.87 | 1.56745 | 240 | 17.8 |
| 60.68 | 1.52495 | 248 | 18.4 |
| 62.067 | 1.49415 | 261 | 19.4 |
| 63.043 | 1.47336 | 262 | 19.5 |
| 65.449 | 1.42489 | 286 | 21.2 |
| 66.425 | 1.40631 | 348 | 25.9 |

### Comparative Example 1

### Preparation of Ti-Al Beta Zeolite on the Basis of M. A. Uguina et al.

The Ti-Al Beta zeolite was prepared in accordance with M. A. Uguina et al., J. Chem. Soc., Chem. Commun., 1994, page 27, as cited in E. G. Derouane et al., “Titanium-substituted zeolite beta: an efficient catalyst in the oxy-functionalisation of cyclic alkenes using hydrogen peroxide in organic solvents”, New. J. Chem., 1998, pages 797-799.

Materials used:

completely ion-free water: 536.90 g

tetraethylammonium hydroxide: 504.90 g

Al(NO₃)₃×9 H₂O: 4.11 g

tetraethyl orthotitanate: 8.11 g

Aerosil 200: 131.20 g

Batch 1: A glass beaker was initially charged with 486.9 g of completely ion-free water. Under agitation at 200 rpm, 504.9 g of tetraethylammonium hydroxide were added and stirred in for 10 min. This is followed by the admixture of 8.11 g of tetraethyl orthotitanate. Following a further 15 min under agitation, 131.2 g of Aerosil were admixed under agitation, and the batch was subsequently stirred for 15 min.

Batch 2: A glass beaker was initially charged with 50 g of completely ion-free water. Under agitation at 200 rpm, 4.11 g of aluminum nitrate were added, and the batch was subsequently stirred for 10 min.

Batch 3: Batch 2 was transferred into batch 1 followed by 15 min of stirring. The liquid gel was pH 13.8 and was placed in an autoclave to crystallize. The temperature in the autoclave was brought to 135° C. in the course of one hour and maintained there for 72 h under autogenous pressure whilst stirring at 100 rpm.

After 72 h, the suspension obtained with a pH of 12.2 is drained off and adjusted with 10% by weight HNO₃ (300 g) to pH 7-8, suction filtered and washed with 10 liters of completely ion-free water. The moist product (filtercake) was subsequently introduced into a porcelain dish, air dried at 120° C. for 10 h and subsequently calcined in air at 580° C. for 4 h. Final weights were 154 g for dried product and 126 g for calcined product.

### Comparative Example 2

### Preparation of MWW Framework Structure Zeolite Comprising Titanium (Ti-MWW)

2.1 Preparation of Boron-Containing MWW (B-MWW)

Deionized water (470 kg) was introduced into a reactor as an initial charge. Under agitation at 70 rpm boric acid (163 kg) was suspended in the water. The suspension was stirred for 3 h. This was followed by the addition of piperidine (273 kg) and stirring of the mixture for a further hour. Colloidal silicon dioxide (Ludox® AS-40, 392 kg) was added to the mixture, the resulting mixture was stirred at 70 rpm for a further hour. The mixture thus obtained was transferred to a crystallization reactor and heated over 5 h to 170° C. under autogenous pressure and agitation at 50 rpm. The temperature of 170° C. was kept substantially constant for 120 h, and during these 120 h the mixture was stirred at 50 rpm. The mixture was then cooled down to 50-60° C. in the course of 5 h. The suspension comprising B-MWW was pH 11.3, determined by measurement with a pH electrode. The B-MWW was separated from this suspension by filtration using a suction filter. The filtercake was then washed with deionized water until the wash liquor had a conductivity of less than 500 microsiemens/cm, The filtercake thus obtained was spray-dried in a spray tower under the following conditions:

Drying gas, nozzle gas: technical-grade nitrogen

Temperature of drying gas:
spray tower temperature (inlet): 270-340° C.
spray tower temperature (outlet): 150-167° C.
filter temperature (inlet): 140-160° C.
scrubber temperature (inlet): 50-60° C.
scrubber temperature (outlet): 34-36° C.

Filter pressure difference: 8.3-10.3 mbar

Nozzle:
uppermost component of nozzle: manufactured by: Gerig; size: 0
nozzle gas temperature: room temperature
nozzle gas pressure: 2.5 bar

Flow pattern mode: nitrogen straight

Construction: dryer-filter-scrubber

Dosing via peristaltic pump: SP VF 15 from Verder Gerig nozzle size:0

Nozzle gas pressure: 1 bar

Gas rate: 1500 kg/h

Filter material: Nomex—needlefelt 20 m²

Throughput rate: about 45 kg/h

The spray tower consisted of a vertically arranged cylinder having a length of 2650 mm and a diameter of 1200 mm, although the cylinder did have conical narrowing at the bottom end. The length of the cone was 600 mm. The atomizing means (a two-component nozzle) was arranged at the top of the cylinder. The spray-dried material was separated from the drying gas in a filter downstream of the spray tower, and the drying gas was then passed through a scrubber. The suspension was passed through the inner opening of the nozzle, and the nozzle gas was passed through the ring-shaped slot surrounding the opening. The spray-dried material was then subjected at 650° C. to a calcination in a rotary calciner having a throughput of 0.5-1.0 kg/h. The calcined material had a boron content of 1.4% by weight, a silicon content of 42% by weight and a total organic carbon content of 0.14% by weight. The crystallinity as determined by x-ray diffraction was 83%, the BET surface area as per DIN 66131 was 462 m²/g and the pore volume determined via mercury porosymmetry as per DIN 66133 was 5.8 mL/g.

2.2 Preparation of Deboronated MWW

a) Deboronation

Water (3750 kg) was passed into a reactor fitted with a reflux condenser. The spray-dried material obtained as per section 2.1 above (125 kg) was suspended in the water under agitation at 40 rpm. Next, the reactor was sealed and the reflux condenser was started up. Stirrer speed was raised to 70 rpm. Under agitation at 70 rpm, the contents of the reactor were heated over 1 h to 100° C. and maintained at 100° C. for 20 h. The contents of the reactor were then cooled down to a temperature of less than 50° C. The resulting deboronated zeolite of framework structure MWW was separated from the suspension by filtration at a nitrogen pressure of 2.5 bar and washed four times with deionized water. After filtration, the filtercake was dried in a nitrogen stream for 6 h.

b) Spray-drying the nitrogen-dried filtercake

The nitrogen-dried filtercake obtained, with the residual moisture content of 77%, as per section 2.2 a) above was used to prepare an aqueous suspension using deionized water. This suspension was spray-dried in a spray tower under the following conditions:

Drying gas, nozzle gas: technical-grade nitrogen

Temperature of drying gas:
spray tower temperature (inlet): 290-310° C.
spray tower temperature (outlet): 140-160° C.
filter temperature (inlet): 140-160° C.
scrubber temperature (inlet): 40-60° C.
scrubber temperature (outlet): 20-40° C.

Filter pressure difference: 6.0-10.0 mbar

Nozzle:
uppermost component of nozzle: manufactured by: Niro, diameter: 4 mm
nozzle gas pressure: 2.5 bar

Flow pattern mode: nitrogen straight

Apparatus used: spray tower with one nozzle

Construction: spray tower-filter-scrubber

Gas rate: 1900 kg/h

Filter material: Nomex® needlefelt 20 m²

Dosing via peristaltic pump: VF 15 (manufacturer: Verder)

The spray tower consisted of a vertically arranged cylinder having a length of 2650 mm and a diameter of 1200 mm, although the cylinder did have conical narrowing at the bottom end. The length of the cone was 600 mm. The atomizing means (a two-component nozzle) was arranged at the top of the cylinder. The spray-dried material was separated from the drying gas in a filter downstream of the spray tower, and the drying gas was then passed through a scrubber. The suspension was passed through the inner opening of the nozzle, and the nozzle gas was passed through the ring-shaped slot surrounding the opening. The spray-dried material had a boron content of 0.06% by weight, a silicon content of 42.5% by weight and a total organic carbon content of <0.2% by weight. The crystallinity as determined by x-ray diffraction was 81%, the BET surface area as per DIN 66131 was 476 m²/g and the pore volume determined via mercury porosymmetry as per DIN 66133 was 4.7 mL/g.

2.3 Preparation of Ti-MWW

Based on the deboronated MWW material obtained as per section 2.2 above, a titanium-containing zeolite of framework structure MWW was prepared as follows:

The deboronated MWW framework structure zeolite obtained as per section 2.2 above (80 kg) was transferred into a first reactor A. Deionized water (225 kg) was transferred into a second reactor B and stirred at 100 rpm, and piperidine (174 kg) was added. During addition, the temperature of the mixture was raised by about 15° C. This was followed by the addition of tetrabutyl orthotitanate (16 kg) and of deionized water (25 kg). Stirring was continued for 60 min. The mixture of reactor B was then transferred into reactor A and stirred at 70 rpm in reactor A, and deionized water (110 kg) was introduced into reactor A and the mixture was transferred from reactor A into reactor B. After stirring at 70 rpm, stirrer speed was lowered to 50 rpm, and the mixture in reactor B was heated to a temperature of 170° C. in the course of 5 h. The temperature of the mixture in reactor B was kept substantially constant at 170° C. under autogenous pressure for 120 h. Then, the suspension comprising Ti-MWW was cooled down to a temperature of less than 50° C. in the course of 5 h. The resulting Ti-MWW zeolite was separated from the suspension by filtration at a nitrogen pressure of 2.5 bar and washed four times with deionized water. After filtration, the filtercake was dried in a nitrogen stream for 6 h. The filtercake obtained was diluted with water and spray-dried in a spray tower under the following conditions:

Drying gas, nozzle gas: technical-grade nitrogen

Temperature of drying gas:
spray tower temperature (inlet): 290-310° C.
spray tower temperature (outlet): 150-170° C.
filter temperature (inlet): 150-170° C.
scrubber temperature (inlet): 30-50° C.
scrubber temperature (outlet): 30-50° C.

Filter pressure difference: 6.0-10.0 mbar

Nozzle:
uppermost component of nozzle: manufactured by: Niro, diameter: 4 mm
nozzle gas pressure: 1.5 bar

Flow pattern mode: nitrogen straight

Apparatus used: spray tower with one nozzle

Construction: spray tower-filter-scrubber

Gas rate: 1800 kg/h

Filter material: Nomex® needlefelt 20 m²

Dosing via peristaltic pump: SP VF 15 (manufacturer: Verder)

The spray tower consisted of a vertically arranged cylinder having a length of 2650 mm and a diameter of 1200 mm, although the cylinder did have conical narrowing at the bottom end. The length of the cone was 600 mm. The atomizing means (a two-component nozzle) was arranged at the top of the cylinder. The spray-dried material was separated from the drying gas in a filter downstream of the spray tower, and the drying gas was then passed through a scrubber. The suspension was passed through the inner opening of the nozzle, and the nozzle gas was passed through the ring-shaped slot surrounding the opening. The spray-dried material had a silicon content of 35% by weight, a titanium content of 2.4% by weight, a total organic carbon (TOC) content of 9.4% by weight and a nitrogen content of 2.4% by weight.

2.4 Acid Treatment of Ti-MWW

The Ti-MWW zeolite obtained as per section 2.3 above was subjected to an acid treatment described in section a) hereinbelow and was subsequently dried as per the spray-drying operation described in section b) hereinbelow, and the spray-dried material is calcined as described in section c) hereinbelow.

a) Acid treatment of spray-dried material obtained as per section 2.3 above

The spray-dried Ti-MWW prepared as per section 2.3 a) above (40 kg) was admixed with nitric acid (30% by weight) (1200 kg) under agitation at 50 rpm. The resulting mixture was stirred for a further 15 min. Stirrer speed was then raised to 70 rpm. The mixture was heated to 100° C. in a reactor as fast as possible and maintained at 100° C. for 20 h under autogenous pressure and agitation. The mixture thus obtained was then cooled down to a temperature of less than 50° C. The cooled mixture was subjected to a filtration, and the filtercake was washed six times with deionized water under nitrogen pressure at 2.5 bar until the wash liquor was pH 7.

b) Spray-drying the mixture of material obtained as per section 2.4 a)

The filtercake obtained as per section 2.4 a) was combined with deionized water to prepare an aqueous suspension. This suspension was spray-dried in a spray tower under the following conditions:

Drying gas, nozzle gas: technical-grade nitrogen

Temperature of drying gas:
spray tower temperature (inlet): 200-330° C.
spray tower temperature (outlet): 140-165° C.
filter temperature (inlet): 140-160° C.
scrubber temperature (inlet): 50-60° C.
scrubber temperature (outlet): 20-40° C.

Filter pressure difference: 7.0-11.0 mbar

Nozzle:
uppermost component of nozzle: manufactured by: Niro, diameter: 4 mm
nozzle gas throughput: 23 kg/h
nozzle gas pressure: 2.5 bar

Flow pattern mode: nitrogen straight

Apparatus used: spray tower with one nozzle

Construction: spray tower-filter-scrubber

Gas rate: 1900 kg/h

Filter material: Nomex® needlefelt 20 m²

Dosing via peristaltic pump: SP VF 15 (manufacturer: Verder)

The spray tower consisted of a vertically arranged cylinder having a length of 2650 mm and a diameter of 1200 mm, although the cylinder did have conical narrowing at the bottom end.

The length of the cone was 600 mm. The atomizing means (a two-component nozzle) was arranged at the top of the cylinder. The spray-dried material was separated from the drying gas in a filter downstream of the spray tower, and the drying gas was then passed through a scrubber. The suspension was passed through the inner opening of the nozzle, and the nozzle gas was passed through the ring-shaped slot surrounding the opening. The spray-dried, acid-treated Ti-MWW material had a silicon content of 40% by weight, a titanium content of 1.5% by weight, a total organic carbon (TOC) content of 1.8% by weight.

c) Calcining the spray-dried material obtained as per section 1.4 b)

The spray-dried material was then subjected at 650° C. to a calcination in a rotary calciner having a throughput of 0.8-1.0 kg/h. The calcined material had a silicon content of 41% by weight, a titanium content of 1.6% by weight and a total organic carbon content of 0.19% by weight. The BET surface area as per DIN 66131 was 438 m²/g. The pore volume determined by mercury porosymmetry as per DIN 66133 was 6.3 mL/g. The crystallinity as determined by x-ray diffraction was 82%.

### Example 2

### Preparation of 2-alkoxycyclohexanol

The zeolites obtained in Example 1 and Comparative Examples 1 and 2 were employed as catalysts in the reaction of cyclohexene with hydrogen peroxide to prepare 2-methoxycyclo-hexanol. The zeolites obtained in Example 1 and Comparative Example 2 were further used as a catalyst in the reaction of cyclohexene with hydrogen peroxide to prepare 2-ethoxycyclo-hexanol. The preparation of 2-methoxycyclohexanol and/or of 2-ethoxycyclohexanol was carried out as follows:

In a reaction vessel, 1.00 g of the particular zeolitic material was mixed with 3.92 g of cyclo-hexene in 20 ml of methanol (cf. #I to #II and #IV to #VII in table 1 below) or, respectively, 20 ml of ethanol (cf. #A and #B in table 2 below). Run #III as per table 1 utilized 0.5 g of the particular zeolitic material. The resulting mixture was admixed with 1.00 g of an aqueous hydrogen peroxide solution (50% by weight of hydrogen peroxide) in the runs as per #I to #VII in table 1 below and in the runs as per #A and #B in table 2 to obtain the loadings (g(H₂O₂)/g(catalyst=zeolitic material) reported in tables 1 and 2. The mixtures were stirred at 65° C. for the times reported in each case in tables 1 and 2.

After removal of the catalyst by filtration and weighing back of the filtrates thus obtained, a sample was taken of each filtrate. The H₂O₂ content of this sample was determined cerimetrically. The filtrate was admixed with sodium sulfite in order to decompose any H₂O₂ still present. A sample was then taken in order to use gas chromatography (GC) analysis to determine the molar amounts of compounds (I) to (V), which potentially form as per the reaction scheme shown below. The molar amounts were used to compute the selectivity of the catalyst as the molar amount of the desired product in the form of 2-methoxycyclohexanol or 2-ethoxycyclohexanol (a compound of formula (I)) based on the molar amount of the mixture of compounds of the formulae (I) to (V).

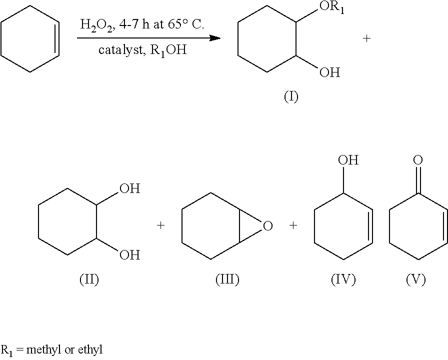

The results obtained in the preparation of 2-methoxycyclohexanol are shown in table 1:

TABLE 1

| | | | Loading/ | |
|---|---|---|---|---|
| | | Reaction | (g(H₂O₂)/g | Selectivity/ |
| # | Catalyst as per | time/h | (catalyst)) | % |
| I | Example 1 | 4 | 0.5 | 93.0 |
| II | Example 1 | 7 | 0.5 | 93.0 |
| III | Example 1 | 4 | 1 | 84.0 |
| IV | Comparative Example 1 | 4 | 0.5 | 81.0 |
| V | Comparative Example 1 | 7 | 0.5 | 75.0 |
| VI | Comparative Example 2 | 4 | 0.5 | 84.5 |
| VII | Comparative Example 2 | 7 | 0.5 | 82.0 |

As is apparent from table 1, the catalyst of the present invention, comprising a zeolitic material of framework structure MWW and comprising B and Ti in the zeolite framework, shows a distinctly higher selectivity than the catalysts of Comparative Examples 1 and 2 for the same reaction time. This is directly apparent from a comparison of #I with #IV and #VI and from a comparison of #II with #V and #VII.

What is more, the catalyst of the present invention did not suffer a loss of selectivity with increasing reaction time, which for example leads to higher conversions; this is apparent from a comparison of #I with #II. By contrast, the catalysts of Comparative Examples 1 and 2 —for identical prolongation of reaction time—suffer in some instances a distinct loss of selectivity; this is apparent from a comparison of #IV with #V and from a comparison of #VI with #VII.

The results obtained in the preparation of 2-ethoxycyclohexanol are shown in table 2:

TABLE 2

| | | | Loading/ | |
|---|---|---|---|---|
| | | Reaction | (g(H₂O₂)/g | Selectivity/ |
| # | Catalyst as per | time/h | (catalyst)) | % |
| A | Example 1 | 4 | 0.5 | 71 |
| B | Comparative Example 2 | 4 | 0.5 | 65 |

These examples show that using the catalyst of the present invention—comprising a zeolitic material of framework structure MWW and comprising B and Ti in the zeolite framework—gives a distinctly higher selectivity than the catalyst of Comparative Example 2.

### SHORT DESCRIPTION OF FIGURES

FIG. 1 shows the ¹¹B solid state NMR spectrum of the zeolite according to Example 1, as measured according to Reference Example 1. The ¹¹B chemical shift (in ppm) is shown on the x-axis, while the intensity (*10⁶) is shown on the y-axis. The scale divisions on the x-axis are, from left to right, at 40, 20, 0, −20. The scale divisions on the y-axis are, from bottom to top, at 0, 1, 2, 3, 4.

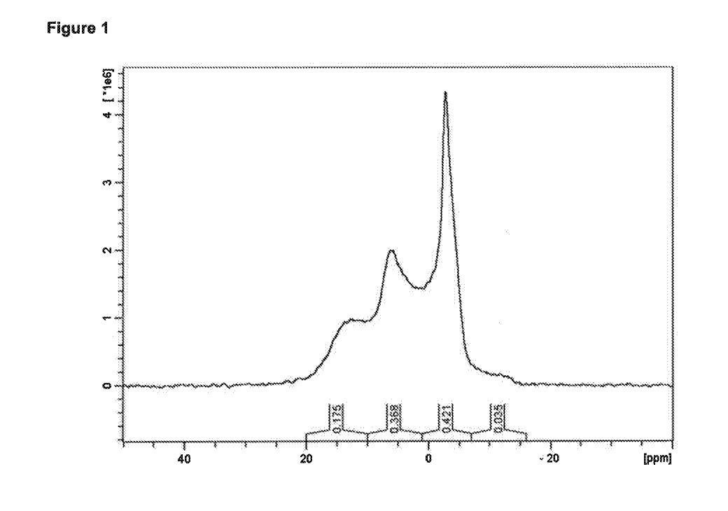

FIG. 2 shows the ²⁹Si solid state NMR spectrum of the zeolite according to Example 1, as measured according to Reference Example 2. The ²⁹Si chemical shift (in ppm) is shown on the x-axis, while the intensity (*10⁶) is shown on the y-axis. The scale divisions on the x-axis are, from left to right, at −90, −100, −110, −120, −130. The scale divisions on the y-axis are, from bottom to top, at 0, 20, 40, 60, 80, 100.

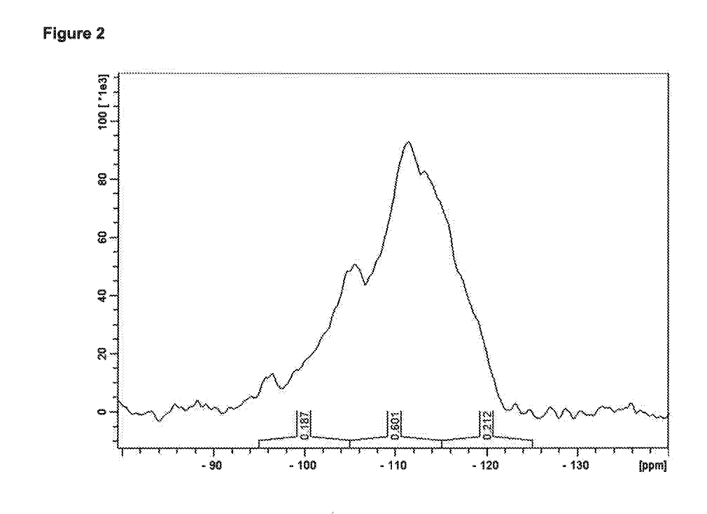

FIG. 3 shows the FT-IR spectrum of the zeolite according to Example 1, as measured according to Reference Example 4. The wavelength (in cm⁻¹) is shown on the x-axis and the extinction is shown on the y-axis. The scale divisions on the x-axis are, from left to right, at 4000, 3500, 3000, 2500, 2000, 1500. The scale divisions on the y-axis are, from bottom to top, at 0.00, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, The wavenumbers indicated on the individual peaks in cm⁻¹ are, from left to right, 3748, 3719, 3689, 3623, 3601, 3536, 1872.

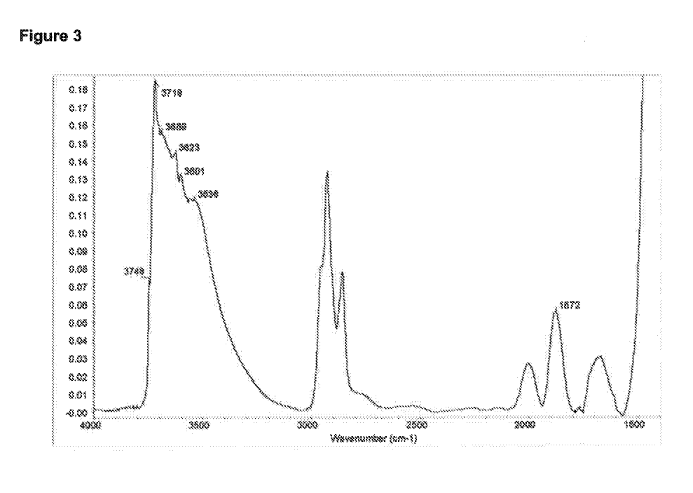

FIG. 4 shows the x-ray diffraction pattern (copper K-alpha radiation) of the zeolite according to Example 1, measured according to Reference Example 5. The degree values (2 theta) are shown on the x-axis and the intensity (Lin (counts)) are shown on the y-axis. The scale divisions on the x-axis are, from left to right, at 2, 10, 20, 30, 40, 50, 60, and 70. The scale divisions on the y-axis are, from bottom to top, at 0 and 3557.

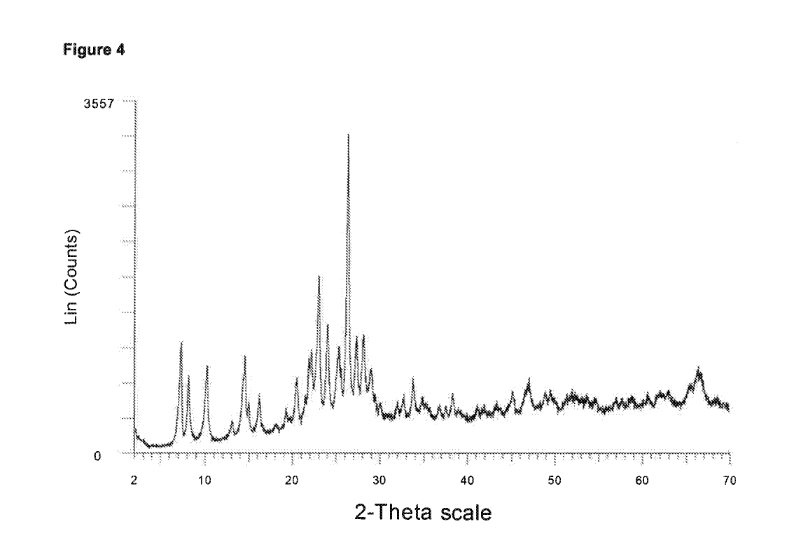

### LITERATURE CITED

GB 2 252 556 A
A. Corma et al., “Activity of Ti-Beta Catalyst for the Selective Oxidation of Alkenes and Alkanes”, Journal of Catalyis (1994), vol. 145, pp. 151-158
Y. Goa et al., “Catalytic Performance of [Ti, Al]-Beta in the Alkene Epoxidation Controlled by the Postsynthetic Ion Exchange”, Journal of Physical Chemistry B 2004, vol. 108, pp. 8401-8411
E. G. Derouane et al., “Titanium-substituted zeolite beta: an efficient catalyst in the oxy-functionalisation of cyclic alkenes using hydrogen peroxide in organic solvents”, New. J. Chem., 1998, pp. 797-799
M. A. Uguina et al., J. Chem. Soc., Chem. Commun., 1994, p. 27

1-15. (canceled)

16. A method of preparing a compound of formula (I) 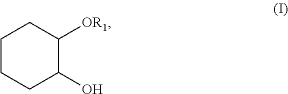
wherein R₁ is alkyl of 1 to 4 carbon atoms, comprising:
(i) providing a liquid mixture comprising cyclohexene, an alcohol R₁OH, hydrogen peroxide and optionally a solvent;
(ii) reacting the cyclohexene with the hydrogen peroxide and the alcohol R₁OH in the mixture provided as per (i) in the presence of a catalyst comprising a zeolitic material of framework structure MWW to obtain a mixture comprising the compound of formula (I), where the framework of the zeolitic material as per (ii) comprises silicon, titanium, boron, oxygen and hydrogen.

17. The method according to claim 16, wherein R₁ is methyl or ethyl, preferably methyl.

18. The method according to claim 16, wherein the liquid mixture provided as per (i) has a molar ratio prior to the reaction as per (ii) of cyclohexene:R₁OH in the range from 1:1 to 1:50, preferably from 1:3 to 1:30 and more preferably from 1:5 to 1:10, and of cyclohexene:hydrogen peroxide in the range from 1:1 to 5:1, preferably from 1.5:1 to 4.5:1, more preferably from 2:1 to 4:1, and while the mass ratio of hydrogen peroxide:zeolitic material of framework structure MWW at the start of the reaction as per (ii) is preferably in the range from 10:1 to 0.1:1, more preferably from 1:1 to 0.2:1, more preferably from 0.75:1 to 0.25:1.

19. The method according to claim 16, wherein the liquid mixture provided as per (i) comprises a solvent, preferably selected from the group consisting of C1-C6-alkyl nitriles, dialkyl ketones of the formula R₂—CO—R₃, where R₂ and R₃ are each independently selected from the group consisting of C1-C6-alkyl, and a mixture of two or more thereof, more preferably from the group consisting of C1-C3-alkyl nitriles, dialkyl ketones of the formula R₂—CO—R₃, where R₂ and R₃ are each independently selected from the group consisting of C1-C3-alkyl, and a mixture of two or more thereof, more preferably from the group consisting of acetonitrile, acetone and a mixture thereof, wherein the molar ratio of solvent:cyclohexene before the reaction as per (ii) is preferably in the range from 20:1 to 1:1, more preferably in the range from 15:1 to 1:1 and more preferably in the range from 10:1 to 1:1, subject to the proviso that where the solvent in said mixture is a mixture of two or more solvents, the molar ratio of solvent:cyclohexene is based on the mixture of said solvents.

20. The method according to claim 16, wherein the mixture provided as per (i) comprises no solvent.

21. The method according to claim 16, wherein the reaction as per (ii) is carried out at a temperature of the liquid mixture in the range from 40 to 150° C., preferably from 50 to 125° C., more preferably from 55 to 100° C., more preferably at the boiling point of the liquid mixture, preferably under reflux.

22. The method according to claim 16, wherein the duration of the reaction as per (ii) is in the range from 1 to 12 h, preferably from 1.5 to 10 h, more preferably from 2 to 8 h.

23. The method according to claim 16, wherein not less than 99% by weight, preferably not less than 99.5% by weight, more preferably not less than 99.9% by weight of the framework of the zeolitic material as per (ii) consists of silicon, titanium, boron, oxygen and hydrogen, wherein the molar ratio of B:Si is preferably in the range from 0.02:1 to 0.5:1, more preferably 0.05:1 to 0.15:1, and the molar ratio of Ti:Si is in the range from 0.01:1 to 0.05:1, preferably 0.017:1 to 0.025:1, and wherein the zeolitic material of framework structure MWW as per (ii) preferably has a boron content, reckoned as elemental B, in the range from 1.0% to 2.0% by weight, more preferably from 1.1% to 1.8% by weight, more preferably from 1.2% to 1.6% by weight, and preferably has a titanium content in the range from 1.0% to 2.0% by weight, more preferably from 1.1% to 1.8% by weight, more preferably from 1.2% to 1.6% by weight, all based on the overall weight of the zeolitic material.

24. The method according to claim 16, wherein the zeolitic material of framework structure MWW as per (ii) is characterized by an x-ray diffractogram having peaks at 2 theta angles of (7.2±0.1)°, (14.5±0.1)°, (22.1±0.1)°, (22.7±0.1)°, (23.0±0.1)°, (24.0±0.1)°, (25.3±0.1)°, (26.3±0.1)°, (27.3±0.1)°, (28.1±0.1)°, wherein the x-ray diffractogram preferably has additional peaks at 2 theta angles of (7.0±0.1)°, (8.1±0.1)°, (10.1±0.1)°, (14.3±0.1)°, (20.4±0.1)°, (21.9±0.1)°, (28.9±0.1)°, (33.8±0.1)°, (47.0±0.1)°, (65.4±0.1)°, (66.4±0.1)°.

25. The method according to claim 16, wherein the zeolitic material of framework structure MWW as per (ii) is obtained as per a method comprising:
(a) providing an aqueous synthesis mixture comprising a silicon source, a boron source, a titanium source and an MWW-templating compound, wherein the temperature of the aqueous synthesis mixture is not more than 50° C.;
(b) heating the aqueous synthesis mixture provided as per (a) from the temperature of not more than 50° C. to a temperature in the range from 160 to 190° C. in the course of a period of at most 24 h;
(c) subjecting the synthesis mixture as per (b) to hydrothermal synthesis conditions under autogenous pressure in a closed-off system at a temperature in the range from 160 to 190° C. to obtain a precursor to the zeolitic material of framework structure MWW in its mother liquor;
(d) separating the precursor to the zeolitic material of framework structure MWW off from its mother liquor; and
(e) calcining the MWW framework structure zeolitic material precursor separated off as per (d) to obtain the zeolite of framework structure MWW.

26. The method according to claim 16, wherein the catalyst as per (ii), preferably in the form of a molding, comprises a binder, preferably a silica binder, in addition to the zeolitic material of framework structure MWW.

27. The method according to claim 16, wherein the mole percentage for the compound of formula (I) in the mixture obtained from the reaction as per (ii), based on the sum total of mole percentages for the compounds of formulae (I), (II), (III), (IV) and (V) 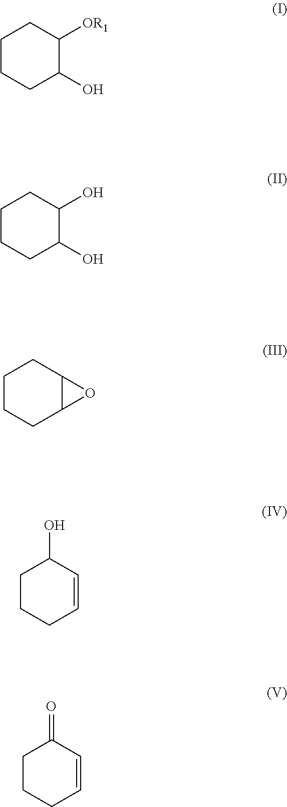
in the mixture obtained from the reaction as per (ii) is not less than 85%, preferably not less than 90%.

28. The method according to claim 16, wherein the mixture obtained from the reaction as per (ii) comprises the compounds of formulae (I) and (II) and optionally at least one of the compounds as per formulae (III), (IV) and (V) 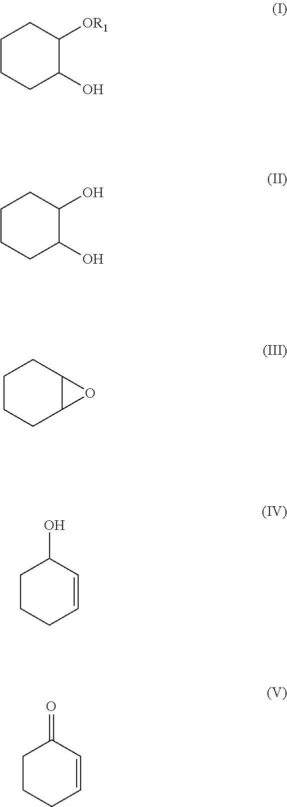
and wherein the mole percentage for the compound of formula (II) in the mixture, based on the sum total of mole percentages for the compounds of formulae (II), (III), (IV) and (V) in the mixture is preferably not less than 70%, more preferably not less than 75%, and more preferably not less than 80%, comprising:
(iii) separating the compound of formula (II) off from the mixture obtained from the reaction as per (ii) to obtain a mixture depleted with respect to the compound of formula (II); and
(iv) optionally separating the compound of formula (I) off from the mixture obtained from the reaction as per (ii) or from the mixture obtained from the separating step as per (iii) as being depleted with respect to the compound of formula (II).

29. A mixture comprising a compound of formula (I) 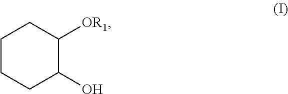
wherein R₁ is alkyl of 1 to 4 carbon atoms, obtained as per the method according to claim 16, wherein the mole percentage for the compound of formula (I) in the mixture, based on the sum total of mole percentages for the compounds of formulae (I), (II), (III), (IV) and (V) 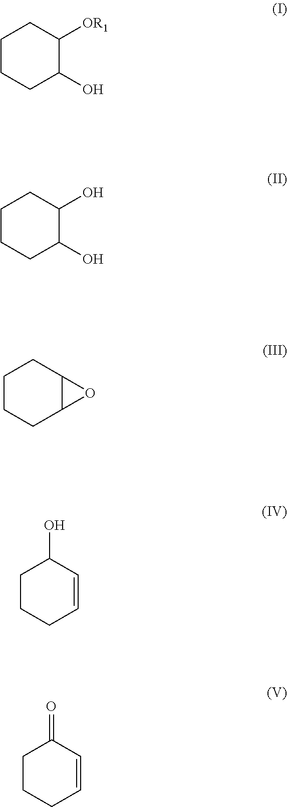
in the mixture is not less than 85%, preferably not less than 90%.

30. A method of using the mixture according to claim 29 in the manufacture of a compound of formula (VI) [chemical structure]
